(19) United States
(12) Patent Application Publication
(10) Pub. No.: US 2019/0183686 A1
(43) Pub. Date: Jun. 20, 2019

(54) Facial Bandage Assembly

(71) Applicant: Mary Farrell, Hutto, TX (US)

(72) Inventor: Mary Farrell, Hutto, TX (US)

(21) Appl. No.: 15/849,036

(22) Filed: Dec. 20, 2017

(51) Int. Cl. A61F 13/12

(52) U.S. Cl. CPC A61F 13/126; A61F 13/00085

(57) ABSTRACT

A facial bandage assembly for absorbing nasal drainage includes a dressing is configured to be positioned on a face such that the dressing extends laterally beneath a nose. The dressing is comprised of a fluid absorbent material to absorb fluid draining from the nose. A strip is positioned within the dressing and the strip is comprised of a resiliently bendable material. The strip is manipulated to shape the dressing into a selected shape to conform to contours of the face. A pair of straps is provided and each of the straps is coupled to the dressing. Each of the straps is extended over a selected one of a pair of ears thereby retaining the dressing beneath the nose.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to bandage devices and more particularly pertains to a new bandage device for nasal drainage.

### BRIEF SUMMARY OF THE INVENTION

An embodiment of the disclosure meets the needs presented above by generally comprising a dressing is configured to be positioned on a face such that the dressing extends laterally beneath a nose. The dressing is comprised of a fluid absorbent material to absorb fluid draining from the nose. A strip is positioned within the dressing and the strip is comprised of a resiliently bendable material. The strip is manipulated to shape the dressing into a selected shape to conform to contours of the face. A pair of straps is provided and each of the straps is coupled to the dressing. Each of the straps is extended over a selected one of a pair of ears thereby retaining the dressing beneath the nose.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front perspective view of a facial bandage assembly according to an embodiment of the disclosure.

FIG. 2 is a back view of an embodiment of the disclosure.

FIG. 3 is a front phantom view of an embodiment of the disclosure.

FIG. 4 is a perspective in-use view of an embodiment of the disclosure.

### DETAILED DESCRIPTION OF THE INVENTION

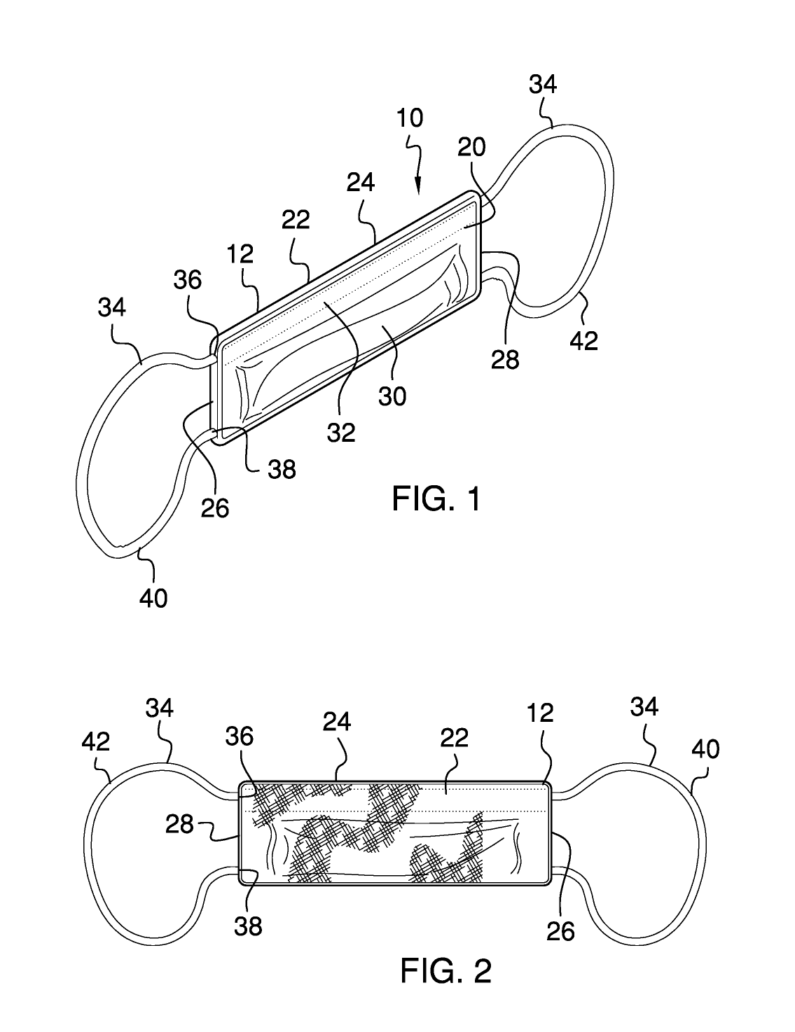

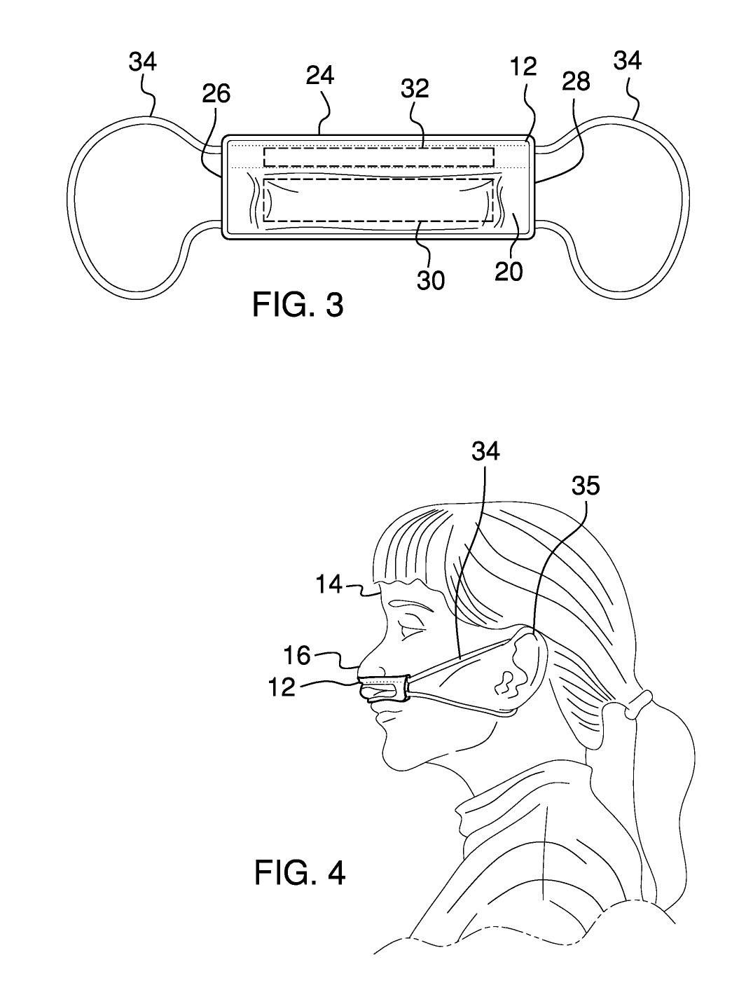

With reference now to the drawings, and in particular to FIGS. 1 through 4 thereof, a new bandage device embodying the principles and concepts of an embodiment of the disclosure and generally designated by the reference numeral 10 will be described.

As best illustrated in FIGS. 1 through 4, the facial bandage assembly 10 generally comprises a dressing 12 that may be positioned on a face 14 such that the dressing 12 extends laterally beneath a nose 16. The dressing 12 is comprised of a fluid absorbent material such as gauze or the like. Thus, the dressing 12 absorbs fluid draining from the nose 16. The fluid may be bodily fluid resulting from a nasal surgical procedure such as a pituitary tumor resection or the like. The dressing 12 has a front side 20, a back side 22 and a peripheral edge 24 extending therebetween. The peripheral edge 24 has a first lateral side 26 and a second lateral side 28 and the dressing 12 is elongated between the first lateral side 26 and the second lateral side 28.

A pad 30 is positioned within the dressing 12. The pad 30 is comprised of a fluid absorbent material such as cotton or the like. Thus, the pad 30 absorbs the fluid draining from the nose 16. The pad 30 is positioned between the front side 20 and the back side 22 of the dressing 12. Moreover, the pad 30 extends substantially between the first lateral side 26 and the second lateral side 28.

A strip 32 is positioned within the dressing 12. The strip 32 is comprised of a resiliently bendable material such as metal or the like. The strip 32 is selectively manipulated to shape the dressing 12 into a selected shape. In this way the dressing 12 is selectively shaped to conform to contours of the face 14. The strip 32 is positioned between the front side 20 and the back side 22 of the dressing 12. Moreover, the strip 32 extends substantially between the first lateral side 26 and the second lateral side 28.

A pair of straps 34 is provided and each of the straps 34 is coupled to the dressing 12. Each of the straps 34 is extended over a selected one of a pair of ears 35 thereby retaining the dressing 12 beneath the nose 16. Each of the straps 34 has a first end 36 and a second end 38 and the pair of straps 34 includes a first strap 40 and a second strap 42. Each of the first 36 and second 38 ends of the first strap 40 is coupled to the first lateral side 26 of the dressing 12 such that the first strap 40 forms a closed loop. Each of the first 36 and second 38 ends of the second strap 42 is coupled to the second lateral side 28 of the dressing 12 such that the second strap 42 forms a closed loop. Moreover, each of the first 40 and second 42 straps is comprised of a resiliently stretchable material such as elastic or the like.

In use, each of the first 40 and second 42 straps is positioned around the associated ear 35 and the dressing 12 is positioned to extend laterally beneath the nose 16. In this way the dressing 12 and the pad 30 absorb the fluid that drains from the nose 16. The strip 32 is manipulated to shape the dressing 12 into the selected shape to keep the dressing 12 abutted against the face 14. In this way the fluid is inhibited from passing between the face 14 and the dressing 12. The dressing 12 is removed and discarded when the dressing 12 becomes soaked with the fluid.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A facial bandage assembly being configured to be worn beneath a nose thereby facilitating said assembly to absorb drainage from the nose, said assembly comprising:
a dressing being configured to be positioned on a face such that said dressing extends laterally beneath a nose, said dressing being comprised of a fluid absorbent material wherein said dressing is configured to absorb fluid draining from the nose;
a strip being positioned within said dressing, said strip being comprised of a resiliently bendable material wherein said strip is configured to be manipulated, said strip shaping said dressing into a selected shape wherein said dressing is configured to conform to contours of the face; and
a pair of straps, each of said straps being coupled to said dressing wherein each of said straps is configured to be extended over a selected one of a pair of ears thereby retaining said dressing beneath the nose.

2. The assembly according to claim 1, wherein said dressing has a front side, a back side and a peripheral edge extending therebetween, said peripheral edge having a first lateral side and a second lateral side, said dressing being elongated between said first lateral side and said second lateral side.

3. The assembly according to claim 2, further comprising a pad being positioned within said dressing, said pad being comprised of a fluid absorbent material wherein said pad is configured to absorb the fluid draining from the nose, said pad being positioned between said front side and said back side of said dressing, said pad extending substantially between said first lateral side and said second lateral side.

4. The assembly according to claim 2, wherein said strip is positioned between said front side and said back side of said dressing, said strip extending substantially between said first lateral side and said second lateral side.

5. The assembly according to claim 2, wherein each of said straps has a first end and a second end, said pair of straps including a first strap and a second strap, each of said first and second ends of said first strap being coupled to said first lateral side of said dressing such that said first strap forms a closed loops.

6. The assembly according to claim 5, wherein each of said first and second ends of said second strap is coupled to said second lateral side of said dressing such that said second strap forms a closed loop, each of said first and second straps being comprised of a resiliently stretchable material.

7. A facial bandage assembly being configured to be worn beneath a nose thereby facilitating said assembly to absorb drainage from the nose, said assembly comprising:
a dressing being configured to be positioned on a face such that said dressing extends laterally beneath a nose, said dressing being comprised of a fluid absorbent material wherein said dressing is configured to absorb fluid draining from the nose, said dressing having a front side, a back side and a peripheral edge extending therebetween, said peripheral edge having a first lateral side and a second lateral side, said dressing being elongated between said first lateral side and said second lateral side;
a pad being positioned within said dressing, said pad being comprised of a fluid absorbent material wherein said pad is configured to absorb the fluid draining from the nose, said pad being positioned between said front side and said back side of said dressing, said pad extending substantially between said first lateral side and said second lateral side;
a strip being positioned within said dressing, said strip being comprised of a resiliently bendable material wherein said strip is configured to be manipulated, said strip shaping said dressing into a selected shape wherein said dressing is configured to conform to contours of the face, said strip being positioned between said front side and said back side of said dressing, said strip extending substantially between said first lateral side and said second lateral side; and
a pair of straps, each of said straps being coupled to said dressing wherein each of said straps is configured to be extended over a selected one of a pair of ears thereby retaining said dressing beneath the nose, each of said straps having a first end and a second end, said pair of straps including a first strap and a second strap, each of said first and second ends of said first strap being coupled to said first lateral side of said dressing such that said first strap forms a closed loops, each of said first and second ends of said second strap being coupled to said second lateral side of said dressing such that said second strap forms a closed loop, each of said first and second straps being comprised of a resiliently stretchable material.
